(19) United States
(12) Patent Application Publication
(10) Pub. No.: US 2018/0199630 A1
(43) Pub. Date: Jul. 19, 2018

(54) ELECTRONIC CIGARETTE

(71) Applicant: Changzhou Patent Electronic Technology Co.,ltd, Changzhou (CN)

(72) Inventor: WEI-HUA QIU, ChangZhou (CN)

(21) Appl. No.: 15/871,099

(22) Filed: Jan. 15, 2018

(30) Foreign Application Priority Data
Jan. 16, 2017 (CN) 201710033864.7

(51) Int. Cl. A24F 47/00; A61M 11/04; A61M 15/06

(52) U.S. Cl. CPC A24F 47/008; A61M 11/042; A61M 2205/3368; A61M 2205/8212; A61M 15/06

(57) ABSTRACT

An electronic cigarette which avoids waste of smoke and the loss of any unnecessary heating energy includes an atomizer, a controller, a sensor, and a prompting device. The atomizer includes an atomizing assembly and a coupled cigarette holder assembly. The cigarette holder assembly includes a cigarette holder, an air flow channel between the cigarette holder and an atomizing cavity of the atomizing assembly, a unidirectional valve mounted in the air flow channel, and the sensor, controller, and prompting device. The user is prompted to inhale smoke only when the available smoke satisfies certain conditions such as temperature, smoke particle density, and humidity. Under a suction force, the unidirectional valve opens an airflow channel the channel being otherwise closed.

### FIELD

The present disclosure relates to simulated smoking technology, and more particularly to an improved electronic cigarette.

### BACKGROUND

Electronic cigarettes produce smoke by heating aerosol-forming substrate for users. It takes some time from heating the aerosol-forming substrate to produce smoke. During this time, since the aerosol-forming substrate being heated unevenly, the aerosol-forming substrate in individual areas will be heated up in advance. The cigarette holder communicates directly with the atomizing cavity, so that the smoke will flow out directly from the cigarette holder. However, at this moment, the smoke volume is small. If the user smokes directly, the volume of smoke is insufficient, but if the user does not take smoke in, the smoke will be wasted. In addition, the smoke that is taken will take away some of the heat energy, thus the rest of unheated aerosol-forming substrate needs more time to be heated up, which reduces the user's experience.

### SUMMARY

The present disclosure provides an electronic cigarette. The electronic cigarette prompts user to smoke when the smoke satisfies certain requirements. The smoke can be drawn and flow out only when a user is inhaling. The electronic cigarette of the present disclosure prevents a waste of smoke and a waste of heat lost caused by a self-flow of the smoke.

The electronic cigarette includes an atomizer, a controller, a sensor, and a prompting device. The atomizer includes an atomizing assembly and a cigarette holder assembly mounted to an end of the atomizing assembly. An atomizing cavity is defined in the atomizing assembly for heating aerosol-forming substrate to generate smoke. The cigarette holder assembly includes a cigarette holder, an airflow channel positioned between the cigarette holder and the atomizing cavity, and a unidirectional valve positioned in the airflow channel.

The sensor is configured to detect certain qualities of the smoke and feed information back to the controller.

A first preset value is pre-stored in the controller. The controller is configured to compare the certain qualities detected with the first preset value, and configured to control the prompting device to transmit a signal when the qualities reach the first preset value.

The reminding device is configured to transmit signal to user according to the controlling of the controller. The signal to user is configured to remind or prompt user to inhale.

Furthermore, when a suction force is applied to the unidirectional valve, the unidirectional valve opens the airflow channel, thus the atomizing cavity communicates with the cigarette holder via the airflow channel. When the suction force disappears, the unidirectional valve closes the airflow channel.

Furthermore, the cigarette holder assembly further includes a cigarette holder base and a cigarette holder upper cover. The cigarette holder is arranged on the cigarette holder upper cover. An end portion of the cigarette holder upper cover opposite to the cigarette holder is connected to the cigarette holder base. The airflow channel includes a smoke outlet channel formed by a space between cigarette holder base and the cigarette holder upper cover.

Furthermore, at least one baffle portion is positioned in the smoke outlet channel. The at least one baffle portion is configured to space the smoke outlet channel to form zigzag channel.

Furthermore, the at least one baffle portion includes a first baffle portion positioned on the cigarette holder base. The airflow channel further includes a vent hole defined on a sidewall of the first baffle portion. The vent hole is in communication with the atomizing cavity and the smoke outlet channel. The unidirectional valve includes a fixing portion fixed to the first baffle portion and a movable portion configured for opening or sealing the vent hole.

Furthermore, the at least one baffle portion further includes a second baffle portion positioned on the cigarette holder base, and a third baffle portion positioned on the cigarette holder upper cover. The third baffle portion is located between the first baffle portion and the second baffle portion.

Furthermore, the cigarette holder further includes a communicating member. The communicating member defines a smoke outlet along the axial direction of the communicating member. A communicating groove is defined on a sidewall of the communicating member along the radial direction of the communicating member, and the communicating groove communicates with the smoke outlet. The airflow channel further includes the smoke outlet and the communicating groove. When the communicating member passes through the cigarette holder base and is coupled to the cigarette holder upper cover, the smoke outlet channel can communicate with the cigarette holder via the communicating groove and the smoke outlet.

Furthermore, a receiving groove is defined on the cigarette holder upper cover. An end of the cigarette holder is rotatably received in the receiving groove. A smoke outlet groove is defined on the cigarette holder. When the cigarette holder is rotated to a receiving position, another end of the cigarette holder is received in the receiving groove, thus the smoke outlet groove is staggered in relation to the smoke outlet. When the cigarette holder is rotated to a suction position, the other end of the cigarette holder is separated from the receiving groove, thus the smoke outlet groove is in communication with the smoke outlet.

Furthermore, the cigarette holder includes a suction portion and a rotating portion positioned at an end of the suction portion. The rotating portion is rotatably connected with an end of the receiving groove. The smoke outlet groove includes a first smoke outlet groove defined on the suction portion and a second smoke outlet groove defined on the rotating portion. The first smoke outlet groove is in communication with the second smoke outlet groove.

Furthermore, the airflow channel further includes a vent groove defined on the cigarette holder base. The vent groove is in communication with the atomizing cavity and the smoke outlet channel. A filter screen is arranged at an end of vent groove adjacent to the atomizing cavity.

Furthermore, the atomizer further includes an outer shell. The cigarette holder assembly is mounted to an end of the outer shell of the atomizing assembly. The controller and the sensor are received in the outer shell. The reminding device is positioned at the outside of the outer shell. The outer shell defines an air inlet communicating with the outside of the outer shell. A vent hole which communicates with the atomizing cavity and the air inlet are defined in the atomizing assembly.

The present disclosure provides a cigarette holder assembly, the cigarette holder assembly is configured for an electronic cigarette.

Furthermore, the cigarette holder assembly includes a cigarette holder, an airflow channel, and a unidirectional valve. The cigarette holder is mounted to an end of the airflow channel, the atomizing cavity is defined at another end of the airflow channel. The unidirectional valve is positioned in the airflow channel. The unidirectional valve is capable of opening the airflow channel to let the cigarette holder communicate with the atomizing cavity via airflow channel. The unidirectional valve is also capable of closing the airflow channel to sever the cigarette holder from the atomizing cavity.

Furthermore, the cigarette holder assembly further includes a cigarette holder base and a cigarette holder upper cover. The cigarette holder is mounted to an end of the cigarette holder upper cover. The cigarette holder base is arranged at another end of the cigarette holder upper cover. The airflow channel further includes a smoke outlet channel formed by the space between the cigarette holder base and the cigarette holder upper cover.

Furthermore, at least one baffle portion is positioned in the smoke outlet channel. The at least one baffle portion is configured to space the smoke outlet channel to form zigzag channel.

Furthermore, the baffle portion includes a first baffle portion positioned on the cigarette holder base. The airflow channel further includes a vent hole defined on a sidewall of the first baffle portion. The vent hole can communicate with the atomizing cavity and the smoke outlet channel. The unidirectional valve includes a fixing portion which is fixed to the first baffle portion and a movable portion. The movable portion is capable of sealing or opening the vent hole.

Furthermore, the at least one baffle portion further includes a second baffle portion positioned on the cigarette holder base and a third baffle portion positioned on the cigarette holder upper cover. The third baffle portion is located between the first baffle portion and the second baffle portion.

Furthermore, the cigarette holder further includes a communicating member. The communicating member defines a smoke outlet along the axial direction. A communicating groove is defined on a sidewall of the communicating member along the radial direction of the communicating member. The airflow channel further includes the smoke outlet and the communicating groove. When the communicating member passes through the cigarette holder base and is coupled to the cigarette holder upper cover, the smoke outlet communicates with the cigarette holder via the communicating groove and the smoke outlet.

Furthermore, a receiving groove is defined on the cigarette holder upper cover. An end of the cigarette holder is rotatably positioned in the receiving groove. A smoke outlet groove is defined on the cigarette holder. When the cigarette holder is rotated to a receiving position, another end of the cigarette holder is received in the receiving groove, thus the smoke outlet groove is staggered in relation to the smoke outlet. When the cigarette holder is rotated to a suction position, the other end of the cigarette holder is separated from the receiving groove, thus the smoke outlet groove can communicate with the smoke outlet.

Furthermore, the cigarette holder includes a suction portion and a rotating portion positioned at an end of the suction portion. The rotating portion is rotatably connected with the receiving groove. The smoke outlet groove includes a first smoke outlet groove defined on the suction portion and a second smoke outlet groove defined on the rotating portion. The first smoke outlet groove communicates with the second smoke outlet groove.

Furthermore, the airflow further includes a vent groove defined on the cigarette holder base. The vent groove communicates with the atomizing cavity and the smoke outlet channel. A filter screen is arranged at an end of the vent groove adjacent to the atomizing cavity.

The unidirectional valve is made of elastic material.

A user is prompted to smoke only when the aerosol-forming substrate and smoke are in certain states. The unidirectional valve can be opened by the smoke only when the user is inhaling. Therein, the vent groove is opened, and the atomizing cavity communicates with the smoke outlet channel to let smoke flow out from the cigarette holder. Compared with a traditional structure, the electronic cigarette of the present disclosure prevents a waste of smoke and a waste of heat lost caused by a self-flow of the smoke.

### BRIEF DESCRIPTION OF THE DRAWINGS

Implementations of the present technology will now be described, by way of example only, with reference to the attached figures.

FIG. 1 is a cross-sectional view of an electronic cigarette.

FIG. 2 is a stereoscopic, exploded view of a cigarette holder assembly of the electronic cigarette of FIG. 1.

FIG. 3 is a schematic view of an electronic cigarette upper cover of the cigarette holder assembly of FIG. 2.

FIG. 4 is a cross-sectional view of a communicating member of the cigarette holder assembly of FIG. 2.

FIG. 5 is a bottom view of the cigarette holder assembly of FIG. 2.

FIG. 6 is an enlarged view of the cigarette holder assembly of the electronic cigarette of FIG. 1.

FIG. 7 is a section view of the electronic cigarette being ready to provide smoke but not being inhaled.

FIG. 8 is a section view of the electronic cigarette in a smoke-ready state and being inhaled.

FIG. 9 is a circuit control block diagram of the electronic cigarette.

### DETAILED DESCRIPTION

Detailed description of specific embodiments of the present application will be given with reference to the accompanying drawings. A number of specific details are set forth in the following description so as to fully understand the present application. However, the present application can be implemented in many ways different from those described herein, and those skilled in the art can make improvements without violating the contents of the present application. Therefore, the present application is not to be considered as limiting the scope of the embodiments described herein.

Several definitions that apply throughout this disclosure will now be presented.

The term “coupled” is defined as coupled, whether directly or indirectly through intervening components, and is not necessarily limited to physical connections. The connection may be such that the objects are permanently coupled or releasably coupled. The term “substantially” is defined to be essentially conforming to the particular dimension, shape, or other feature that the term modifies, such that the component need not have that exact feature. The term “comprising,” when utilized, means “including, but not necessarily limited to”; it specifically indicates open-ended inclusion or membership in the so-described combination, group, series, and the like.

When an element is considered to be “fixed to” another element, it can be either directly fixed on another element or indirectly fixed on another element with a centered element. When an element is considered to be “coupled with” another element, it can be either directly coupled with another element or indirectly coupled with another element.

Unless otherwise defined, all technical and scientific terms used herein have the same meaning as commonly understood by one skilled in the art. The terms used in specification of the present application are only for describing specific embodiments, and are not intended to limit the present application. The terms “and/or” used herein includes any and all combinations of one or more of associated listed items.

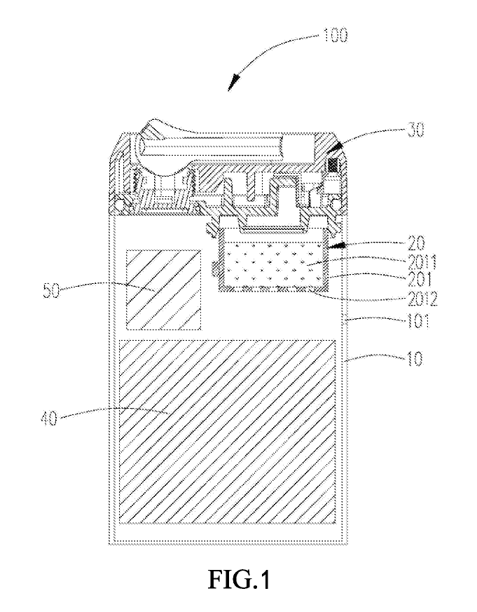

Referring to FIG. 1, the present disclosure provides an electronic cigarette 100. The electronic cigarette 100 is configured to heat aerosol-forming substrate, such as tobacco liquid, tobacco leaf, to form smoke for the user to inhale. The electronic cigarette 100 includes an atomizer (not labeled in FIGS), a battery 40, and a controller 50. The atomizer includes an outer shell 10, an atomizing assembly 20 fixed within the outer shell 10, and a cigarette holder assembly 30 detachably mounted on the upper portion of the outer shell 10. The battery 40 and the controller 50 are received in the outer shell 10. The electronic cigarette 100 can be configured to heat the aerosol-forming substrate. In an alternative embodiment, the battery 40 and the controller 50 can be positioned in shells other than the outer shell 10.

The outer shell 10 is substantially a cylindrical structure with an opening at the top end. To allow the external air follow into the electronic cigarette 100, an air inlet 101 is defined on a sidewall of the outer shell 10.

The atomizing assembly 20 includes a bowl 201. The bowl 201 is substantially a cylindrical structure with an opening at the top end thereof. An atomizing cavity 2011 is defined in the bowl 201. The atomizing cavity 2011 is configured to receive the aerosol-forming substrate. The aerosol-forming substrate received in the atomizing cavity 2011 is heated to form smoke. At least one vent hole 2012 is defined on the bottom portion of the bowl 201. The vent hole 2012 is in communication with the atomizing cavity 2011. The vent hole 2012 is in communication with the air inlet 101 of the outer shell 10. The external air flows into the atomizing cavity 2011, via through the air inlet 101 and the vent hole 2012, and then mixed with the smoke in the atomizing cavity 2011.

The bowl 201 is configured as a heating member to heat the aerosol-forming substrate. The bowl 201 is electrically connected with the controller 50. In the illustrated embodiment, the bowl 201 is a cylindrical stainless steel heater. In an alternative embodiment, the bowl 201 can be a cylindrical ceramic heater. In an alternative embodiment, the bowl 201 is configured to store the aerosol-forming substrate only, and a separate heater for heating the aerosol-forming substrate is positioned in the bowl 201.

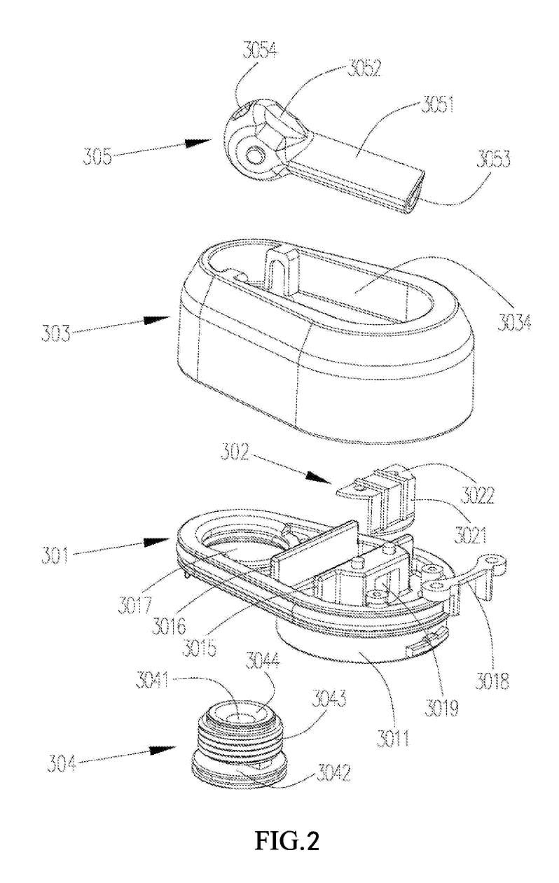

Referring to FIG. 2, the cigarette holder assembly 30 includes a cigarette holder base 301, a unidirectional valve 302, a cigarette holder upper cover 303, a communicating member 304, and a cigarette holder 305.

The cigarette holder base 301 is detachably arranged on the top end of the outer shell 10. The cigarette holder upper cover 303 is arranged on the top end of the cigarette holder base 303 via the communicating member 304. The unidirectional valve 302 is mounted to the cigarette holder base 301, and the unidirectional valve 302 is configured to control the opening and closing of airflow. A user can inhale smoke by the cigarette holder 305 which is mounted to the cigarette holder upper cover 303.

The cigarette holder base 301 is substantially a disk-like structure. The bottom end of the cigarette holder base 301 extends downwardly to form a connecting portion 3011 corresponding to the bowl 201. The connecting portion 3011 is detachably connected with the bowl 201 via threads. In an alternative embodiment, the connecting portion 3011 can be detachably connected with the bowl 201 via latching connection, magnetic connection, plugging connection, and so on.

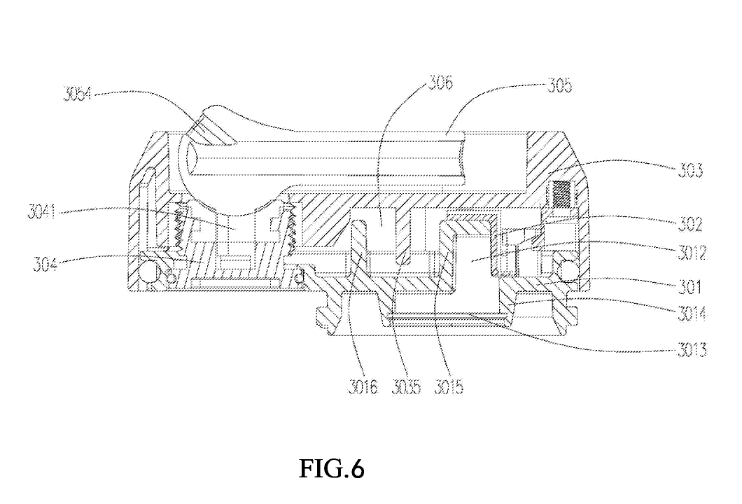

Referring to FIGS. 5 and 6, the cigarette holder base 301 defines a vent groove 3012 communicating with the atomizing cavity 2011. A filter screen 3013 is positioned at an end of the vent groove 3012. In the illustrated embodiment, the bottom end portion of the cigarette holder base 301 extends downwardly to form an installing portion 3014. The installing portion 3014 is in communication with the vent groove 3012. The filter screen 3013 is arranged at the lower end portion of the installing portion 3014, to prevent aerosol-forming substrate from being carried out with and wasted by the airflow.

Referring to FIG. 2, the top end portion of the cigarette holder 301 extends upwardly to from a first baffle portion 3015 and a second baffle portion 3016 arranged apart from the first baffle portion 3015. There is an interval between the first baffle portion 3015 and the second baffle portion 3016. The first baffle portion 3015 and the second baffle portion 3016 are substantially plate-like structures. In the illustrated embodiment, the first baffle portion 3015 is positioned corresponding to the vent groove 3012, and a vent hole 3019 is defined on a sidewall of the first baffle portion 3015 away from the second baffle portion 3016. The vent hole 3019 is in communication with the vent groove 3012.

A first installing hole 3017 is defined on a side of the cigarette holder base 301. A first latching member 3018 is positioned on a side of the cigarette holder base 301 opposite to the first installing hole 3017. The first baffle portion 3015 and the second baffle portion 3016 are located between the first installing hole 3017 and the first latching member 3018. The first baffle portion 3015 is adjacent to the first latching member 3018. The second baffle portion 3016 is adjacent to the first installing portion 3017. The first installing hole 3017 is an unthreaded hole. The first latching member 3018 includes two longitudinal bars (not labeled in the figures) and a cross bar (also not labeled). The two longitudinal bars are opposite to each other, and fixed to the cigarette holder base 301. The cross bar is fixed to the longitudinal bars and positioned between the two longitudinal bars. The longitudinal bars and the cross bar form substantially a “ ” shaped structure.

The unidirectional valve 302 is substantially a “_{┐}” shaped sheet structure. The unidirectional valve 302 includes a movable portion 3021 and a fixing portion 3022 positioned on the top end of the movable portion 3021. The fixing portion 3022 is fixed to the top end of the first baffle portion 3015. The movable portion 3021 attaches to a sidewall of the first baffle portion 3015 and corresponds to the vent hole 3019. The movable portion 3021 can move away from the first baffle portion 3015 and can automatically move back toward the first baffle portion 3015. When the unidirectional valve 302 is in an initial state, the movable portion 3021 attaches to the first baffle portion 3015 and seals the vent hole 3019. When the unidirectional valve 302 is deformed, the movable portion 3021 moves away from the first baffle portion 3015 and opens the vent hole 3019.

In the illustrated embodiment, a pair of embossments (not labeled in the figures) are positioned on the top portion of the first baffle portion 3015. The fixing portion 3022 defines through holes (also not labeled) corresponding to the embossments. The through holes of the fixing portion 3022 are coupled to the embossments to fix the location of unidirectional valve 302.

In order to achieve the deformation and automatic repositioning, the unidirectional valve 302 is made of elastic material. In the illustrated embodiment, the unidirectional valve 302 is made of silica gel.

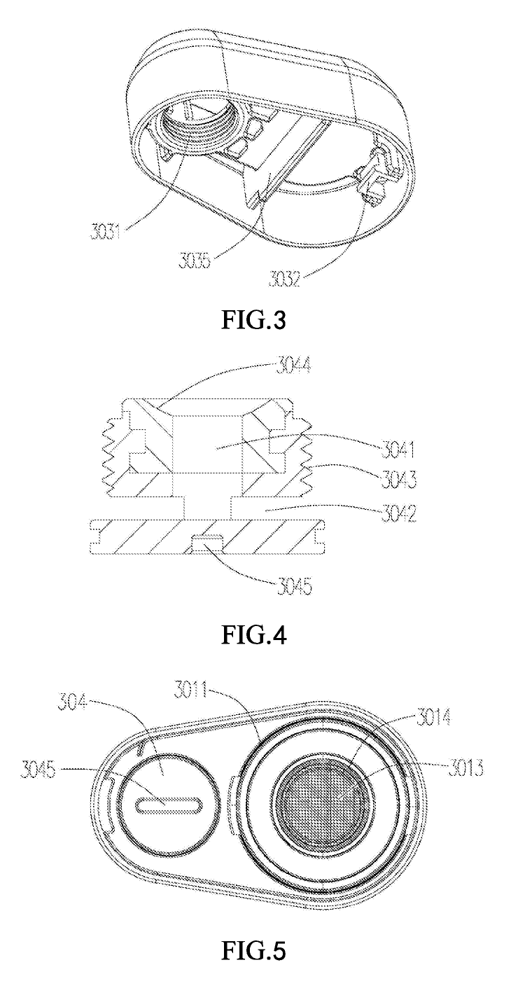

Referring to FIG. 3, the cigarette holder upper cover 303 is substantially a box structure with an opening at the bottom end thereof. A second installing hole 3031 corresponding to the first installing hole 317 is defined on the cigarette holder upper cover 303. A second latching member 3032 corresponding to the first latching member 3018 is positioned on the cigarette holder 303. The second installing hole 3031 is a threaded hole. The second latching member 3032 includes a latching portion configured to be latched to the cross bar of the first latching member 3018, so that the cigarette holder upper cover 303 can be latched to the cigarette holder base 301. In the illustrated embodiment, the first latching member 3018 and the second latching member 3021 are made of plastic materials.

Referring to FIG. 2, a receiving groove 3034 is defined on the top end of the cigarette holder upper cover 303, to receive the cigarette holder 305. An end of the receiving groove 3034 is in communication with the second installing hole 3031.

Referring to FIG. 3, the top portion of the cigarette holder upper cover 303 extends downwardly along the axial direction to form a third baffle portion 3035. The third baffle portion is substantially plate-like structures. Referring to FIG. 6, when the cigarette holder upper cover 303 is connected to the cigarette holder base 301, the third baffle portion 3035 is parallel with the first baffle portion 3015 and the second baffle portion 3016, and the third baffle portion 3035 is positioned at a substantially middle portion between the first baffle portion 3015 and the second baffle portion 3016. A space between the cigarette holder upper cover 303 and the cigarette holder base 301 defines a smoke outlet channel 306. The unidirectional valve 302 is located at an end of the smoke outlet channel 306. The smoke outlet channel 306 is spaced by the first baffle portion 3015, the second baffle portion 3016, and the third baffle portion 3035, to form zigzag channel. The smoke flows to the cigarette holder 305 along the zigzag channel, thereby cooling the smoke to prevent the smoke from overheating and avoid scalding. In an alternative embodiment, the number of the baffle portion is not limited to three and may be one, two, or more than three. For example, a fourth baffle portion can be positioned on the cigarette holder upper cover 303. The fourth baffle portion can be located between the third baffle portion 3035 and the second baffle portion 3016.

Referring to FIG. 2 and FIG. 4, the communicating member 304 is a substantially columnar structure. The top end portion of the communicating member 304 defines a smoke outlet 3041 along an axial direction of the communicating member 304. A communicating groove 3042 is defined on an external wall of the communicating member 304 along the radial direction of the communicating member 304. The communicating groove 3042 is in communication with the smoke outlet 3041. In the illustrated embodiment, an external thread 3043 is defined at the sidewall of the communicating member 304 above the communicating groove 3042. When the communicating member 304 is installed to a certain position, the external thread 3043 is matched with the second installing hole 3031, and the part of the communicating member 304 below the communicating groove 3042 is received in the first installing hole 3017. At this moment, an end of the smoke outlet channel 306 away from the unidirectional valve 302 is in communication with the smoke outlet 3041 via the communicating groove 3042.

In the illustrated embodiment, an arcuate groove 3044 is defined at the top end of the communicating member 304. The arcuate groove 3044 is in communication with the smoke outlet 3041.

Referring to FIG. 4 and FIG. 5, a detaching portion 3045 is positioned at the bottom end of the communicating member 304 to allow for detaching of the communicating member 304. In the illustrated embodiment, the detaching portion 3045 is a groove defined on a lower surface of the communicating member 304. A coin, metal piece or screwdriver can be inserted into the groove to unscrew the communicating member 304, thus the cigarette holder assembly 30 can be detached for changing accessories or cleaning accessories. In addition, as coins, metal pieces are more common, so users do not need to purchase additional disassembly tools.

Referring to FIG. 2, the cigarette holder 305 includes a suction portion 3051 and a rotating portion 3052 fixed at an end of the suction portion 3051. A smoke outlet groove (not labeled in the figures) is positioned in the cigarette holder 305, and connects the suction portion 3051 with the rotating portion 3052. The smoke outlet groove includes a first smoke outlet groove 3053 defined along the axial direction of the suction portion 3051 and a second smoke outlet groove 3054 defined at the rotating portion 3052. The second smoke outlet groove 3054 is in communication with the first smoke outlet groove 3053.

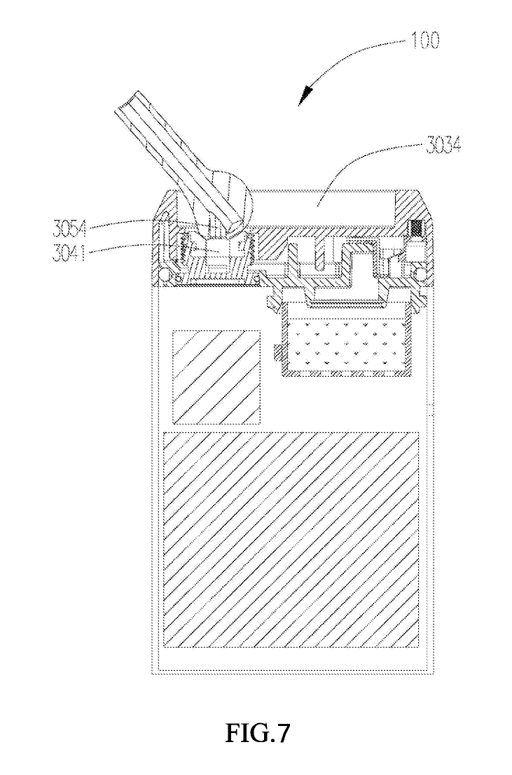

The rotating portion 3052 is rotatably mounted in an end of the receiving groove 3034 adjacent to the second installing hole 3031. At least a part of an outer contour of the rotating portion 3052 forms an arcuate surface (not labeled in the figures). The second smoke outlet groove 3054 intersects the arcuate surface, and the arcuate surface is attached to a sidewall of the arcuate groove 3044 of the communicating member 304. During the process of rotating the cigarette holder 305, the arcuate surface of the rotating portion 3052 is tightly attached to the sidewall of the arcuate groove 3044. Referring to FIG. 6, When the cigarette holder 305 is rotated until it is fully received in the receiving slot 3034, (the cigarette holder 305 being in a receiving position in this state), the second smoke outlet groove 3054 is staggered in relation to the smoke outlet 3041 of the communicating member 304, and thus not in communication with the smoke outlet 3041. Referring to FIG. 7, when the cigarette holder 305 is rotated until it is stopped by a sidewall of the receiving groove 3034 adjacent to the second installing hole 3031 (the cigarette holder 305 being in a suction position in this state), the second smoke outlet groove 3054 is in communication with the smoke outlet 3041. Therefore, the user can now smoke. When the electronic cigarette 100 is not in use, the cigarette holder 305 can be received in the receiving groove 3034, to reduce the overall volume of the electronic cigarette 100 for easy carrying and shipping.

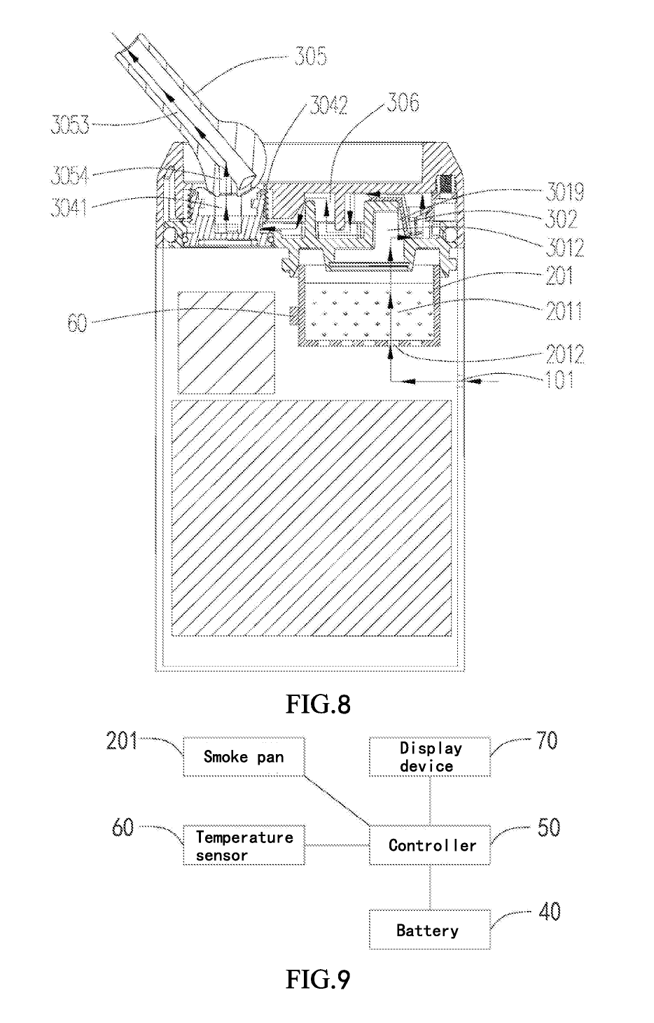

Referring to FIG. 8, when the user wants to smoke, the cigarette holder needs to be rotated to the suction position, then the user holds the cigarette holder 305 for inhaling. A negative pressure is generated in the smoke outlet channel 306, acting on the movable portion 3021 of the unidirectional valve 302, and the movable portion 3021 moves toward an inner portion of the smoke outlet channel 306 or moves away from the first baffle portion 3015. Therefore, the vent hole 3019 is opened, and the vent groove 3012 is in communication with the smoke outlet channel 306. External air passes through the air inlet 101 and the vent hole 2012 to enter the atomizing cavity 2011. The external air mixes with the smoke in the atomizing cavity 2011, then the mixed gas can flow to the user via the vent groove 3012, the vent hole 3019, the smoke outlet channel 306, the communicating groove 3042, the smoke outlet 3041, the second smoke outlet groove 3054, and the first smoke outlet groove 3053. The direction of arrows shown in FIG. 8 shows a direction of smoke/airflow to the user.

When the user stops inhaling, the air pressure in the smoke outlet channel 306 is restored. The unidirectional valve 302 resets to seal the vent hole 3019, so that a connection between the smoke outlet channel 306 and the atomizing cavity 3011 is cut off, and smoke cannot flow. On the other side, when the user stops inhaling, the external air cannot flow into the atomizing cavity 2011 via cigarette holder 302, which avoids a waste of the smoke and a loss of heat. Furthermore, when the user is using the electronic cigarette 100, his hand holds the outer shell 10 tightly, and the air inlet 101 defined on the outer shell 10 may be very close to the hand. If the smoke is forced by the external air to vent from the air inlet 101, the hand of user may be burned. The unidirectional valve 302 prevents such burning.

The vent groove 3012, the vent hole 3019, the smoke outlet channel 306, the communicating groove 3042 and the smoke outlet 3041 form an airflow channel communicating with the atomizing cavity 2011 and the cigarette holder 302. In the illustrated embodiment, the unidirectional valve 302 is positioned between the vent hole 3019 and the smoke outlet channel 3016. In the other embodiments, the unidirectional valve 302 can be positioned at other positions of the airflow channel. For example, the unidirectional valve 302 can be positioned in the vent groove 3012, or positioned between the communicating groove 3042 and the cigarette holder 302, or positioned in the smoke outlet channel 306, or positioned between the smoke outlet channel 306 and the communicating groove 3042, or positioned between the communicating groove 3042 and the smoke outlet 3041, or positioned in the smoke outlet 3041.

In the illustrated embodiment, the unidirectional valve 302 is substantially a bending structure. The unidirectional valve 302 and the first baffle portion 3015 cooperate to control the opening and closing of airflow. The structure of the unidirectional valve 302 can be adjusted accordingly. For example, when the unidirectional valve is positioned in the vent groove 3012, the unidirectional valve 302 can be a plate structure. A sidewall of the vent groove 3012 can extend along the radial direction of the vent groove 3012 to form a supporting portion. An end of the unidirectional valve 302 can be fixed, and another end of the unidirectional valve 3012 can be movable. The fixed end of the unidirectional valve 302 can be fixed to the sidewall of the vent groove 3012 opposite to the supporting portion. The movable end of the unidirectional valve 302 can be supported by the supporting portion. When the unidirectional valve is in an initial state, the movable end of the unidirectional valve 302 is supported by the supporting portion, and the unidirectional valve 302 seals the vent groove 3012. When the unidirectional valve 302 is deformed, the movable end is away from the supporting portion of the vent groove 3012, so that the vent groove 3012 is opened.

FIG. 9 shows a control circuit of the electronic cigarette 100. The electronic cigarette 100 of the present disclosure further includes a temperature sensor 60 and a display device 70. The display device 70 is located outside of the outer shell 10. The controller 50 is electrically connected to the battery 40, the bowl 201, the temperature sensor 60, and the display device 70. The temperature sensor 60 is configured to detect a temperature of the smoke. When the battery 40 supplies power for the bowl 201, a temperature of the atomizing cavity 2011 rises gradually. The aerosol-forming substrate in the atomizing cavity 2011 is heated to produce smoke. The temperature sensor 60 detects the temperature of the smoke in real-time, and feeds signal back to the controller 50. A first preset value is pre-stored in the controller 50. The controller 50 compares the temperature of the smoke with the first preset value. When the temperature of the smoke reaches the first preset value, the controller 50 prompts the user to smoke via a signal displayed on the display device 70.

In the illustrated embodiment, the first preset value is a temperature of the smoke when the aerosol-forming substrate is sufficiently atomized. The first preset value can be a default set value pre-stored in the controller 50, or it can be input by user to the controller based on the actual experience of the user.

In an alternative embodiment, the first preset value can be a desired temperature of a user or a recommended temperature as a default set temperature stored in the controller 50. The temperature of smoke when the aerosol-forming substrate is atomized, the desired temperature of the user, and the recommended temperature can be equal to each other or different from each other. If the temperature of smoke is 300° C. when the aerosol-forming substrate is atomized sufficiently, however a user feels best when the temperature is 250° C., the desired temperature of smoke can be set at 250° C. The first preset value can be set as 250° C. by the user.

In other embodiments, other states or qualities of smoke can be detected by other types of sensors. By comparing the detected qualities with pre-stored parameters, a determination can be made as to whether a user should be prompted to smoke.

For example, one parameter of smoke can be a humidity of the smoke. A humidity sensor can replace the temperature sensor 60 or can be additional, and can be electrically connected to the controller 50. The humidity sensor can be positioned in the airflow channel or the atomizing cavity 2011. Accordingly, the first preset value can be a humidity value. The humidity value can be a humidity when the aerosol-forming substrate is atomized sufficiently, or a desired humidity of a user, or a recommended humidity of smoke. The humidity sensor detects the humidity of smoke and feeds signal back to the controller 50. The controller 50 compares the humidity of smoke with the first preset value. When the humidity of smoke reaches the first preset value, the controller 50 prompts the user to smoke via a signal displayed on the display device 70.

For another example, a parameter can be a particle concentration of smoke. An ion smoke detector or a photoelectric smoke detector can replace the temperature sensor 60 or be additional, and can be electrically connected to the controller 50. The ion smoke detector or the photoelectric smoke detector can be positioned in the airflow channel or the atomizing cavity 2011. Accordingly, the first preset value can be a particle concentration value. The particle concentration value can be a particle concentration when the aerosol-forming substrate is atomized sufficiently, a desired particle concentration of a user, or a recommended particle concentration of smoke. The ion smoke detector or the photoelectric smoke detector detects the particle concentration of smoke and feeds signal back to the controller 50. The controller 50 compares the particle concentration of smoke with the first preset value. When the particle concentration of smoke reaches the first preset value, the controller 50 prompts the user to smoke.

In the illustrated embodiment, a second preset value is also pre-stored in the controller 50. The second preset value is greater than the first preset value. The controller 50 also compares the temperature of smoke with the second preset value. When the temperature of smoke reaches the second preset value, the controller 50 controls the battery 40 to stop supplying power to the bowl 201, to avoid the temperature of atomizing cavity 2011 being too high and the aerosol-forming substrate being over atomized. Therefore, a suitable temperature of smoke is between the first preset value and the second preset value. The control circuit can prevent the user from smoking the aerosol-forming substrate which is insufficiently atomized or over atomized. In an alternative embodiment, when the parameter is the humidity of smoke, the second preset value also can be set to control the humidity of smoke between the first preset value and the second preset value. Therefore, the smoke will not be too dry or too wet. When the parameter is the particle concentration of smoke, the second preset value also can be set to control the particle concentration of smoke between the first preset value and the second preset value, to prevent the smoke from being too thick or being too thin.

Referring to FIG. 8, in the illustrated embodiment, the temperature sensor 60 is positioned on an external wall of the bowl 201. In an alternative embodiment, the temperature sensor can be embedded in a wall of the bowl 201, or posted on an internal wall of the bowl 201, or positioned in the bowl 201, (being positioned in the atomizing cavity 2011), or received in the atomizing cavity, or positioned in the airflow channel.

In an alternative embodiment, the electronic cigarette 100 can prompt user to smoke by other device which indicates a prompting. The other device can include at least one of a display device, one or more indication lights, a vibration device, and an audible device. For example, an indication color or a number of bright lights can be used to remind users. Vibration can be used to remind users when the other device includes a vibration device. An audible sound can be used to remind users when the other device includes a loudspeaker.

The disclosed electronic cigarette reminds the user to smoke when certain qualities of smoke (such as temperature) reach the first preset value, improving user experience. Only when the user is actually inhaling does the unidirectional valve 302 deform. The vent hole 3019 is opened, and the atomizing cavity 2011 is in communication with the smoke outlet channel 306, so that the smoke, previously captive, can flow out from the cigarette holder 305. Compared with a traditional structure, the electronic cigarette of the present disclosure prevents a waste of smoke and a waste of heat lost caused by a self-flow of the smoke.

The embodiments shown and described above are only examples. Many details are often found in the art such as the other features of atomizers. Therefore, many such details are neither shown nor described. Even though numerous characteristics and advantages of the embodiments have been set forth in the foregoing description, together with details of the structure and function of the embodiments, the present disclosure is illustrative only, and arrangement of parts within the principles of the embodiments to the full extent indicated by the broad general meaning of the terms in which the appended claims are expressed.

What is claimed is:

1. An electronic cigarette, comprising:
an atomizer, comprising an atomizing assembly, an atomizing cavity being defined in the atomizing assembly, for heating aerosol-forming substrate to generate smoke,
a sensor configured to detect a related physical parameter of smoke,
a controller configured to get feedback from the sensor and compare the related physical parameter of smoke with a pre-stored first preset value,
wherein when the related physical parameter of smoke reaches the first preset value, the smoke is suitable to release.

2. The electronic cigarette as claimed in claim 1, wherein the atomizer further comprises a cigarette holder assembly, the cigarette holder assembly comprises a cigarette holder, an airflow channel positioned between the cigarette holder and the atomizing cavity, and a unidirectional valve positioned in the airflow channel, when a suction force is applied to the unidirectional valve, the unidirectional valve opens the airflow channel, thus the atomizing cavity communicates with the cigarette holder via the airflow channel, and the smoke is released.

3. The electronic cigarette as claimed in claim 2, further comprising a reminding device, wherein when the related physical parameter of smoke reaches the first preset value, the reminding device is controlled by the controller to transmit a reminding signal, the reminding signal is configured to remind users to inhale.

4. The electronic cigarette as claimed in claim 2, wherein the cigarette holder assembly further comprises a cigarette holder base and a cigarette holder upper cover, the cigarette holder is arranged on the cigarette holder upper cover, an end portion of the cigarette holder upper cover opposite to the cigarette holder is connected to the cigarette holder base, the airflow channel comprises a smoke outlet channel formed by a space between cigarette holder base and the cigarette holder upper cover.

5. The cigarette holder as claimed in claim 4, wherein at least one baffle portion is positioned in the smoke outlet channel, the at least one baffle portion are configured to space the smoke outlet channel to form zigzag channel.

6. The electronic cigarette as claimed in claim 4, wherein the cigarette holder further comprises a communicating member, the communicating member defines a smoke outlet along an axial direction of the communicating member, a communicating groove is defined on a sidewall of the communicating member along a radial direction of the communicating member, and the communicating groove is communicated with the smoke outlet, the airflow channel further comprises the smoke outlet and the communicating groove, when the communicating member passes through the cigarette holder base and is coupled to the cigarette holder upper cover, the smoke outlet channel is communicated with the cigarette holder via the communicating groove and the smoke outlet.

7. The electronic cigarette as claimed in claim 6, wherein a receiving groove is defined on the cigarette holder upper cover, an end of the cigarette holder is rotatably positioned in the receiving groove, a smoke outlet groove is defined on the cigarette holder, when the cigarette holder is rotated to a receiving position, another end of the cigarette holder is received in the receiving groove, thus the smoke outlet groove is staggered to the smoke outlet, when the cigarette holder is rotated to a suction position, the other end of the cigarette holder is separated from the receiving groove, thus the smoke outlet groove is communicated with the smoke outlet.

8. The electronic cigarette as claimed in claim 7, wherein the cigarette holder comprises a suction portion and a rotating portion positioned at an end of the suction portion, the rotating portion is rotatably connected with an end of the receiving groove, the smoke outlet groove comprises a first smoke outlet groove defined on the suction portion and a second smoke outlet groove defined on the rotating portion, the first smoke outlet groove is communicated with the second smoke outlet groove.

9. The electronic cigarette as claimed in claim 4, wherein the airflow channel further comprises a vent groove defined on the cigarette holder base, the vent groove is communicated with the atomizing cavity and the smoke outlet channel respectively, a filter screen is arranged at an end of vent groove adjacent to the atomizing cavity.

10. The electronic cigarette as claimed in claim 1, wherein the atomizer further comprises an outer shell, the outer shell defines an air inlet communicated with the outside of the outer shell, a vent hole communicated with the atomizing cavity and the air inlet is defined in the atomizing assembly.

11. A cigarette holder assembly configured for an electronic cigarette having an atomizing cavity, comprising:
an airflow channel,
a cigarette holder mounted to an end of the airflow channel, and
a unidirectional valve positioned in the airflow channel,
wherein the atomizing cavity is defined at another end of the airflow channel, the unidirectional valve is capable of opening the airflow channel, to let the cigarette holder communicate with the atomizing cavity via the airflow channel, the unidirectional valve is also capable of closing the airflow channel, to partition the cigarette holder from the atomizing cavity.

12. The cigarette holder assembly as claimed in claim 11, wherein the cigarette holder assembly further comprises a cigarette holder base and a cigarette holder upper cover, the cigarette holder is mounted to an end of the cigarette holder upper cover, the cigarette holder base is arranged at another end of the cigarette holder upper cover, the airflow channel further comprises a smoke outlet channel formed by a space between the cigarette holder base and the cigarette holder upper cover.

13. The cigarette holder assembly as claimed in claim 12, wherein at least one baffle portion is arranged in the smoke outlet channel, the at least one baffle portion is configured to space the smoke outlet channel to form zigzag channel.

14. The cigarette holder assembly as claimed in claim 13, wherein the at least one baffle portion comprises a first baffle portion positioned on the cigarette holder base, the airflow channel further comprising a vent hole defined on a sidewall of the first baffle portion, the vent hole is in communication with the atomizing cavity and the smoke outlet channel respectively, the unidirectional valve comprises a fixing portion fixed to the first baffle portion and a movable portion configured for sealing or opening the vent hole.

15. The cigarette holder assembly as claimed in claim 14, wherein the at least one baffle portion further comprising a second baffle portion positioned on the cigarette holder base and a third baffle portion positioned on the cigarette holder upper cover, the third baffle portion is located between the first baffle portion and the second baffle portion.

16. The cigarette holder assembly as claimed in claim 12, wherein the cigarette holder further comprises a communicating member, the communicating member defines a smoke outlet along an axial direction of the communicating member, a communicating groove is defined on a sidewall of the communicating member along a radial direction of the communicating member, the airflow channel further comprises the smoke outlet and the communicating groove, when the communicating member passes through the cigarette holder base and is coupled to the cigarette holder upper cover, the smoke outlet is communicated with the cigarette holder via the communicating groove and the smoke outlet.

17. The cigarette holder assembly as claimed in claim 16, wherein a receiving groove is positioned on the cigarette holder upper cover, an end of the cigarette holder is rotatably positioned in the receiving groove, a smoke outlet groove is defined on the cigarette holder, when the cigarette holder is rotated to a receiving position, another end of the cigarette holder is received in the receiving groove, thus the smoke outlet groove is staggered to the smoke outlet, when the cigarette holder is rotated to a suction position, the other end of the cigarette holder is separated from the receiving groove, thus the smoke outlet groove is communicated with the smoke outlet.

18. The cigarette holder assembly as claimed in claim 17, wherein the cigarette holder comprises a suction portion and a rotating portion positioned at an end of the suction portion, the rotating portion is rotatably connected with the receiving groove, the smoke outlet groove comprises a first smoke outlet groove defined on the suction portion and a second smoke outlet groove defined on the rotating portion, the first smoke outlet groove is communicated with the second smoke outlet groove.

19. The cigarette holder assembly as claimed in claim 12, wherein the airflow further comprises a vent groove defined on the cigarette holder base, the vent groove is communicated with the atomizing cavity and the smoke outlet channel respectively, a filter screen is arranged at an end of the vent groove adjacent to the atomizing cavity.

20. The cigarette holder assembly as claimed in claim 11, wherein the unidirectional valve is made of elastic material.
